(19) United States
(12) Patent Application Publication
(10) Pub. No.: US 2006/0142650 A1
(43) Pub. Date: Jun. 29, 2006

(54) Systems and methods for medical interventional optical monitoring with molecular filters

(75) Inventors: Robert A. Lodder, Nicholasville, KY (US); John Carberry, Talbott, TN (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 11/184,809

(22) Filed: Jul. 20, 2005

Related U.S. Application Data
(60) Provisional application No. 60/589,437, filed on Jul. 20, 2004.

(51) Int. Cl. A61B 5/00

(52) U.S. Cl. 600/342; 600/310

(57) ABSTRACT

A medical interventional optical monitoring system for determining the geometry and composition of a lumen is provided. An absorption spectrum is obtained through the use of molecular filters and a switching assembly. In particular, an absorption spectrum can be obtained by reflecting a light beam from a catheter against a lumen wall or material, and the resulting spectrum is passed through one or more filters having a specified absorption spectrum defined by a single atom or compound. If the reflected sample spectrum contains the wavelengths of the absorption spectrum of the filter, the sample did not absorb the wavelengths and does not contain the substance. Alternatively, the light can be filtered prior to entry of the light into the catheter. The apparatus includes a switching assembly that sequentially places one or more filters into the light path to determine if the subject atom or compound is contained in the lumen.

This application claims the benefit of U.S. Provisional Application No. 60/589,437 filed Jul. 20, 2004, which is hereby incorporated by reference in its entirety.

### BACKGROUND OF THE INVENTION

1. Field of Invention

This invention pertains to an optical monitoring system. More particularly, this invention pertains to an optical monitoring system that compares an absorption spectrum of a filter with that obtained from a sample or specimen. The system uses a switching assembly enabling a plurality of filters to be used for the comparison.

2. Description of the Related Art

It is desirable to identify chemical and/or biological agents, and other constituents particularly, in fast moving streams or in remote locations of the body. Examples where such identification is desired include femoral, renal, carotid, and coronary passages. In these cases, successful interrogation can benefit from very quick identification of a number of predetermined constituents.

What is needed is a system and method for determining the constituents of a sample or specimen.

### BRIEF SUMMARY OF THE INVENTIONS

According to one embodiment of the present invention, a medical intervention optical monitoring system for determining the constituents of a sample or specimen is provided. An absorption spectrum obtained from a reflection inside a lumen is passed through one or more filters having a specified absorption spectrum defined by a single atom, molecule, or compound. If the filter's absorption spectrum is included in the sample's absorption spectrum, then the sample is determined to contain that atom or compound. For example, if the reflected sample spectrum contains the wavelengths of the absorption spectrum of the filter, that means the sample did not absorb the wavelengths and the sample does not contain the substance. The apparatus includes a switching assembly that sequentially places one or more filters into the light path to determine if the subject atom or compound is contained in the sample.

In one embodiment, the switching assembly includes a 1×N switch distributing an optical signal to one of several filters with each filter monitored by a photodetector. In another embodiment, the switching assembly includes a first 1×N switch distributing an optical signal to one of several filters. The outputs of the filters are input to a second 1×N switch that switches the signals from the filters to a single photodetector. In still another embodiment, the switching assembly includes a rotary switch that directs the optical signal through one of several filters and into a photodetector. In one such embodiment, the optical signal is directed through a rotating prism, through stationary filters that are located radially around the prism, and into the photodetectors on the opposite side of the filters. In another such embodiment, the optical signal is directed through each of several filters mounted on a rotating disk. The filters rotate around a stationary prism and intercept the optical signal as it travels from the prism to a photodetector.

In one embodiment, the optical monitoring system includes an examination fiber contained in a catheter that is adapted for moving along the wall of a lumen, such as an artery, to interrogate the composition of sample material encountered therein. Light can be manipulated by the use of an optical switch at either or both of the ingress or egress of the examination fiber to provide a very high rate of interrogation by a relatively large field of inspection elements.

One embodiment of the invention provides a system for optically evaluating the composition of a sample, such as a biological specimen, that includes: at least one molecular absorption filter having an absorption spectrum at least substantially specific for a preselected atom or preselected compound; a detector configured to measure the intensity of light transmitted through the at least one molecular absorption filter; and a processor operably linked to the detector and configured to determine, based on the amount that the molecular absorption filter reduces the intensity of light in the sample spectrum, whether the atom or compound is present in the sample and/or an extent to which the atom or compound is present in the sample.

A related embodiment of the invention provides a system for optically evaluating the composition of a sample, such as a biological specimen, that includes: at least two molecular absorption filters; each molecular absorption filter having an absorption spectrum at least substantially specific for a preselected atom or preselected compound; and the absorption spectrums of at least two of the molecular absorption filters being at least substantially specific for different atoms or compounds; a light distributing element configured to distribute at least part of a sample spectrum of light produced by illuminating a sample with a light source through each of the at least two molecular absorption filters; at least one detector configured to measure the intensity of light transmitted through each of the molecular absorption filters; and a processor operably linked to the detector and configured to determine, based on the amount that the molecular absorption filter reduces the intensity of light in the sample spectrum, whether the atom or compound for that filter is present in the sample and/or an extent to which the atom or compound for that filter is present in the sample.

A further embodiment of the invention provides a system for optically evaluating whether an in vivo biological sample has a preselected abnormal condition, that includes: at least one molecular absorption filter having an absorption spectrum at least substantially specific for a preselected atom or preselected compound, the presence, absence, or extent of the atom or compound in the sample being associated, alone or in combination with the presence, absence, or extent of one or more other analytes, with a preselected abnormal tissue condition, such as an atherosclerotic condition of a blood vessel; a detector configured to measure the intensity of light transmitted through the at least one molecular absorption filter; and a processor operably linked to the detector and configured to determine (by way of executable computer instructions): based on the amount that the molecular absorption filter reduces the intensity of light in the sample spectrum whether the atom or compound is present in the sample and/or an extent to which the atom or compound is present in the sample; and based at least in part on whether the atom or compound is present in the sample and/or on the extent to which the atom or compound is present in the sample, whether the sample has the preselected abnormal tissue condition.

One embodiment of the invention provides a method for optically evaluating the composition of a sample, such as a biological specimen, that includes the steps of: illuminating a sample with light from a light source to produce a resulting sample spectrum of light; directing at least part of the sample spectrum through at least one molecular absorption filter having an absorption spectrum at least substantially specific for a preselected atom or preselected compound; measuring the intensity of light transmitted through the at least one molecular absorption filter; and determining, based on the amount that the molecular absorption filter reduces the intensity of light in the sample spectrum, whether the atom or compound is present in the sample and/or an extent to which the atom or compound is present in the sample.

A related embodiment of the invention provides a method for optically evaluating the composition of a sample, such as a biological specimen, that includes the steps of: illuminating a sample with light from a light source to produce a resulting sample spectrum of light; directing the sample spectrum through each of at least two molecular absorption filters; each molecular absorption filter having an absorption spectrum at least substantially specific for a preselected atom or preselected compound; and the absorption spectrums of at least two of the molecular absorption filters being at least substantially specific for different atoms or compounds; measuring the intensity of light transmitted through each of the molecular absorption filters; and for each of the molecular absorption filters, determining, based on the amount that the molecular absorption filter reduces the intensity of light in the sample spectrum, whether the atom or compound for the filter is present in the sample and/or an extent to which the atom or compound for the filter is present in the sample.

A further embodiment of the invention provides a method for optically evaluating whether an in vivo biological sample has a preselected abnormal condition that comprises the steps of: illuminating a biological sample in vivo with light from a light source to produce a resulting sample spectrum of light; directing at least part of the sample spectrum through at least one molecular absorption filter having an absorption spectrum at least substantially specific for a preselected atom or preselected compound, the presence, absence, or extent of the atom or compound in the sample being associated, alone or in combination with the presence, absence, or extent of one or more other analytes, with a preselected abnormal condition, such as an atherosclerotic condition of a blood vessel; measuring the intensity of light transmitted through the at least one molecular absorption filter; determining, based on the amount that the molecular absorption filter reduces the intensity of light in the sample spectrum, whether the atom or compound is present in the sample and/or an extent to which the atom or compound is present in the sample; and determining, based at least in part on whether the atom or compound is present in the sample and/or on the extent to which the atom or compound is present in the sample, whether the sample has the preselected abnormal condition.

The invention also provides correspondingly related embodiments in which one or more molecular absorption filters are, for example, placed between outgoing light from the light source and the sample, rather than between the incoming sample absorption spectrum and the detector(s).

### BRIEF DESCRIPTION OF THE DRAWINGS

The above-mentioned features of the invention will become more clearly understood from the following detailed description of the invention read together with the drawings in which:

FIG. 1 is a simplified block diagram of one embodiment of the present invention;

FIG. 2 is a block diagram of another embodiment of the present invention;

FIG. 3 is a diagram of one embodiment of the switching mechanism; and

FIG. 4 is a diagram of another embodiment of the switching mechanism.

### DETAILED DESCRIPTION OF PREFERRED EMBODIMENTS

FIG. 1 is a simplified block diagram of one embodiment of an optical monitoring system 100, an apparatus for determining the constituents of a sample. A light source 102 is directed toward and reflected from a specimen 104. The light beam, after reflecting from the specimen 104, passes through a filter 118 and into an optical detector 120. The output of the optical detector 120 is input to a processor 122.

The light source may, for example, be a broad-spectrum source; that is, the spectrum is at least substantially continuous. In one such embodiment, the source includes a tungsten filament. The light source may, for example, be either a tunable laser or a laser emitting an at least substantially continuous spectrum between upper and lower wavelength limits. In still another embodiment, the light source includes a broad-spectrum source and a band-gap filter, which is an optical filter that selects certain spectra for passage, and rejects others. A non-continuous spectrum light source may, for example, also be used, so long as there is suitable overlap between the absorption spectrum of the molecular absorption filter and the light provided from the source.

Light from the light source 102 is then passed through an image field of the specimen 104, which is examined by the light from the source 202 as a function of the specimen's 104 reflection, absorption, transmission, or diffraction within the field.

The specimen 104 absorbs certain wavelengths of the light from the source 102 and produces an absorption spectrum. In an absorption spectrum, portions of a continuous spectrum (light containing all wavelengths) are missing because they have been absorbed by the medium through which the light has passed; the missing wavelengths appear as dark lines or gaps when viewing the absorption spectrum. This is contrasted with an emission spectrum, which consists of all the radiations emitted by atoms or molecules of an incandescent material. The missing portions of an absorption spectrum provide information as to the makeup of the specimen 104 because the missing portions correspond to the constituents of the specimen 104 that absorb the missing wavelengths.

In one embodiment, the filter 118 is a molecular filter, or molecular absorption filter; that is, a filter with such a construction that it produces an absorption spectrum based upon a single atomic element or molecule. Molecular absorption filters suitable for use according to the invention may, for example, be chemical-based molecular absorption filters, or solid-state molecular absorption filters, or a combination thereof may be used. Those skilled in the art will appreciate that the absorption spectrum for a chemical-based molecular absorption filter for an atom or compound of interest can be at least substantially transposed to provide a corresponding solid-state molecular absorption filter. Furthermore, one or more filters can be used. From the incoming light source 102, the molecular filter 118 absorbs the spectral lines that correspond to the absorption spectrum of the filter's 118 filtering material. Another characteristic of the filter 118 is the transmittance; that is, the amount of light that is transmitted through the filter 118. Transmittance is typically expressed as a percentage. A lower transmittance results in a greater intensity reduction over the spectrum.

Placing a molecular filter 118 in the light path containing an absorption spectrum from the specimen 104 will have different results depending on whether the material for which the filter is specific is present in the specimen. If the material producing the absorption spectrum of the filter 118 is contained in the specimen, then there will not be a reduction of the light passing through the filter 118. The filter 118 does not reduce the light intensity because the absorption spectrum of the filter 118 has all its elements in common with the absorption spectrum of the specimen 104. However, if the material producing the absorption spectrum of the filter 118 is not contained in the specimen, then there will be a reduction of the light passing through the filter 118. The reduction in the intensity of light passing through both the specimen 104 and the filter 118 is due to the filter 118 absorbing wavelengths passed by the specimen 104. In this embodiment, the photodetector 120 is responsive to the wavelengths of interest; that is, the photodetector 120 is sensitive to the light intensity over a wavelength range that encompasses the absorption spectra containing the information used to determine the constituents of the specimen 104.

More generally, the magnitude by which the molecular absorption filter reduces the intensity of light (that remains after interaction with the specimen) is proportional to the extent to which the specimen absorbs wavelengths characteristic of the absorption spectra of the material for which the filter is specific. Thus, if a specimen contains the material for which the molecular absorption filter is specific, the magnitude by which the molecular absorption filter reduces the intensity of light (that remains after interaction with the specimen) will be proportional to the extent (e.g., the relative or absolute amount or concentration) of the material in the specimen. Those skilled in the art will readily recognize that a system according to the invention can be calibrated and configured to determine whether a subject material is present or absent in a specimen, is present above or below a selected threshold amount or concentration in a specimen, and/or to determine a relative or absolute amount or concentration of the material in a specimen.

The sensing and characterizing of spectral absorption, reflection, transmission, and/or diffraction with regard to wavelengths, band-pass gaps, and other spectral energy of various characterizations allows identification of both the geometry and composition of materials within those fields of spectral absorption, reflection, transmission, and/or diffraction.

As used herein, the processor 122 should be broadly construed to mean any computer or component thereof that executes software. The processor 122 includes a memory medium that stores software, a processing unit that executes the software, and input/output (I/O) units for communicating with external devices. Those skilled in the art will recognize that the memory medium associated with the processor 122 can be either internal or external to the processing unit of the processor without departing from the scope and spirit of the present invention.

The processor 122 should be broadly construed to mean any computer or component thereof that executes software. In one embodiment, the processor 122 is a general purpose computer; in another embodiment, it is a specialized device for implementing the functions of the invention. Those skilled in the art will recognize that the processor 122 includes an input component, an output component, a storage component, and a processing component. The input component receives input from external devices, such as the optical detector 120. If the external devices, such as the optical detector 120, have an analog device, in one embodiment, the input component includes an analog-to-digital converter (ADC) for converting the analog input signal to a digital signal used by the processor 122. The output component sends output to external devices, such as a display unit and a printer. The storage component stores data and program code (computer instructions). In one embodiment, the storage component includes random access memory. In another embodiment, the storage component includes non-volatile memory, such as floppy disks, hard disks, and writeable optical disks. The processing component executes the instructions included in the software and routines.

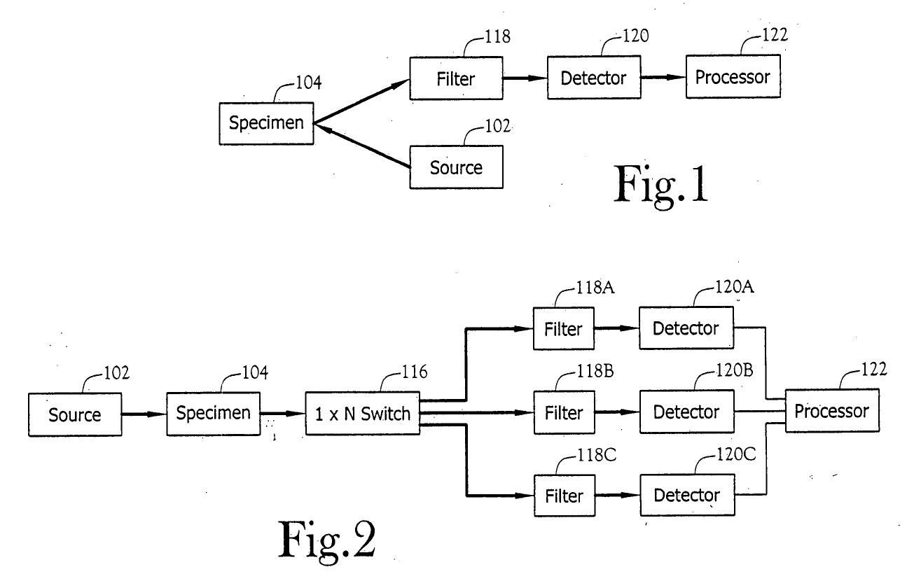

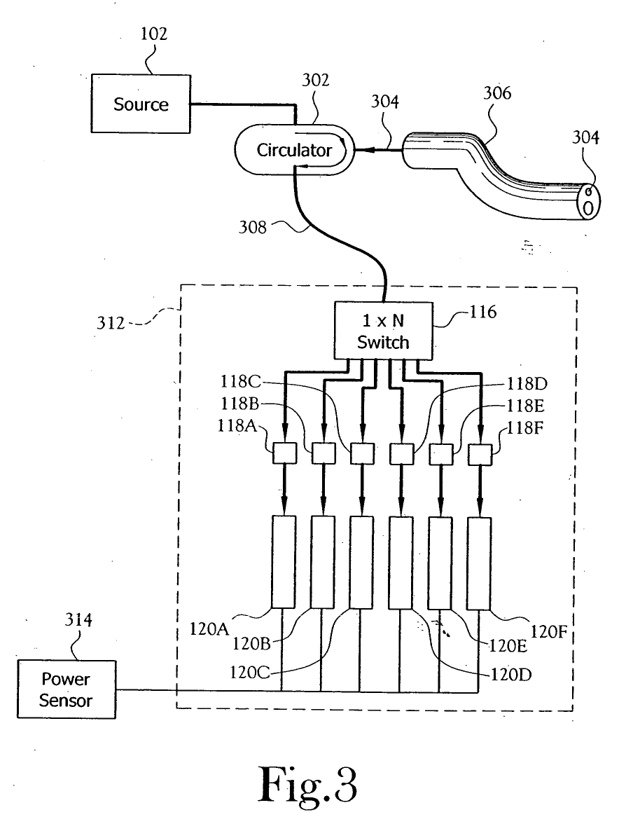

FIG. 2 is a block diagram of another embodiment of the present invention. In the embodiment illustrated in FIG. 1, a light source 102 is reflected from a specimen 104. In the embodiment illustrated in FIG. 2, a light source 102 passes through a specimen 104. In either case, the resulting light beam, which is the absorption spectrum of the specimen 104, passes through a 1×N switch 116 that directs the light beam through one of several pairs of filters 118 and detectors 120. By operating the switch 116 to select each of the filters 118A, 118B, 118C sequentially in rapid succession, the specimen 104 is quickly screened for containing one of the materials represented by the molecular filter 118. If the specimen 104 is a flowing or moving material, the illustrated embodiment allows for real-time monitoring and screening of the specimen 104 for specific materials. In such a case, the switching speed of the switch 116 is such that the absorption spectrum from the specimen 104 that is monitored represents a small volume of the specimen 104, but that volume is monitored with such a frequency that results returned are representative to the real-time concentration of constituents of the specimen 104.

In the illustrated embodiment, the 1×N switch 116 is shown as a 1×3 switch. Those skilled in the art will recognize that the number of ports (N) on the switch 116 must be at least as great as the number of filters 118 desired to be used.

In one embodiment, one of the filters 118A is a neutral density filter that passes the complete spectrum. The output of the neutral density filter 118A provides a reference to compare to the output of the other filters 118B, 118C. In various other embodiments, one or more of the filters 118 is a single or cascaded Bragg grating or a thin film filter.

Identification of a number of constituents non-invasively of a specimen 104 may be performed by a molecular factor computation system (MFCS) or a principal component analysis (PCA) for each constituent, where each MFCS or PCA is quickly switched by an optical switch 116 through and out of an optical fiber, allowing a variety of constituents to be interrogated within a very short period. For each preselected constituent, a molecular factor component (MFC) can be quickly calculated using an optical switch 116, molecular factor filters 118, and single optical fibers, allowing a variety of constituents to be interrogated with a very short duty cycle.

Integrated computational imaging (ICI) is the abstraction of data from physical fields and encoded instream to produce meaningful information. Both spectral and spatial data is recorded and encoded into meaningful information. Further, the use of very wide spectrum or wavelengths, or wide band-gaps, called hyperspectral integrated computational imaging (HICI), provides the basis for using molecular filters 118 for near instant identification of constituents, or materials, preselected by the filters 118. Such information, in various embodiments, is a function of transmission, reflectance, diffraction, and/or absorption of the source 102 by the specimen 104 being interrogated.

Lenslet arrays, masks, filters, and detectors of various types are employed to encode spatial or spectral features of an HICI. Both MFCS and PCA are used to create spectrometer functions that produce factor scores, in the case of MFCS at the detector, which allow, in combination with optical switches, the rapid remote interrogation for a number of factors.

Given a set of training spectra collected at all available wavelengths, it is possible to rationally select molecular filter (MF) materials to perform PCA, which maximizes the signals from the spectral regions with the most variability by most heavily weighting them in calibration. However, principal component loadings heavily weight signals in the positive and negative direction, which cannot be done with molecular filters without an offsetting signal gained at one wavelength with a signal lost at another wavelength. Because only absolute values are represented with molecular filters, two molecular filters are needed for a principal component, one for the positive loadings and one for the negative loadings. The molecular filter materials are selected by examining the sample spectra. The transmission spectrum of the molecular filter material is as similar as possible to the absolute value of the loadings spectrum being targeted.

Both PCA and MFC are used to create spectrometer functions that produce factor scores. However, only MFC completes PCA at the detector without a computer, which allows, through optical switches, the rapid remote interrogation of a sample for a number of factor scores. The molecules in each molecular filter effectively compute the calibration function by weighting the signals received at each wavelength over a broad wavelength range. Each molecular filter is a correlate for identifying a material of interest, be it a biological agent or a chemical entity.

The MFC computing molecules are selected by comparing the spectrum of prospective molecular filter materials to the loadings spectra calculated by PCA. Given a set of training spectra collected at all available wavelengths, or at least those of interest for the molecular filter system being considered, one rationally selects molecular filter materials to perform PCA. Using a conventional spectrometer, mixtures of liquid molecular filters can be titrated to produce the optimum PC result. Digital libraries of the spectra of a variety of candidate filters can be examined in reference to the training spectra by spectral matching software.

Increasing the number of wavelengths in calibration and prediction increases the specificity and produces a model less susceptible to spectral noise if the right wavelengths and weightings are selected. Both PCA and MFC are calibrated to measure constituents of interest while ignoring most interferences, and both are applied to analysis of complex systems, because only calibration information on the constituents of interest is necessary and considered.

A molecular factor computation system (MFCS) differs from PCA in that molecular absorption filters provide information relating to the spatial and spectral features of an HICI and are used as mathematical factors in spectral encoding to create a factor analytic optical calibration in a high throughput spectrometer. Also, PCA is slower than MFC, which uses molecular filters that correspond directly to sample constituents. Molecular absorption filters 118 are used as mathematical factors in spectral encoding to create a factor-analytic optical calibration in a high-throughput spectrometer. The molecular filters 118 compute the calibration function by weighting the signals received at each wavelength over a broad wavelength range. One or two molecular filters 118 are oftentimes sufficient to produce a detector voltage that is proportional (directly or inversely, depending on the bias of a detector) to an analyte concentration in the image field. Each filter 118 is a correlate for identifying a material of interest, be it a biological agent or a chemical entity.

MFC computing molecules for the molecular filters 118 are selected by comparing the spectrum of prospective filter materials to the loadings spectra calculated by PCA. Given a set of training spectra collected at all available wavelengths, or at least those of interest for the molecular filter system being considered, one rationally selects molecular filter 118 materials to perform PCA. PCA is designed to maximize the signals from the spectral regions with the most variability by most heavily weighting them in calibration. The spectrum of the filter materials should be as similar as possible to the absolute value of the loadings spectrum being targeted. Using a conventional spectrometer, mixtures of liquid molecular filters are titrated to produce the optimum PC result. In one embodiment, digital libraries of the spectra of a wide variety of candidate filters are examined in reference to the training spectra by image recognition software.

Using more wavelengths or a broadband light source provides a good averaging effect that produces a model less susceptible to spectral noise. Both PCA and MFC are calibrated to measure constituents of interest while ignoring most interferences, and both are applied to complex analysis and systems, because only calibration information on the constituents of interest is necessary and considered.

Variations in the spectrum from a sample 104 are due to several factors, including the differences in the specimen constituents, interactions between constituents, and overall absorbance. In molecular computing, the molecular filters are selected to maximize the integrated differences in the variation-spectra within a certain band-pass. In PCA, the variation-spectra are used in place of the raw spectral data for constructing the calibration model. The variation-spectra are used to reconstruct the original spectrum of a certain sample by multiplying each variation-spectrum by a unique constant scaling factor and summing the results until the new spectrum agrees with the unknown spectrum. The fraction of each spectrum that must be added to reconstruct the unknown spectral data is associated with the concentration of the constituents.

In PCA, the spectra of the variations are termed eigenvectors, or loadings, spectral loadings, loading vectors, or principal components or factors, based on the means used to compute the spectra. Eigenvectors are related to loadings. The scaling factors employed to reconstruct the individual spectra are called scores. Ordinary spectroscopy and PCA chemometrics record signals with a narrow band-pass at each wavelength and then weights the signals a at each wavelength λ with a coefficient f
score=f₁α_{λ1}+f₂α_{λ2}+f₃α_{λ3}+f₄α_{λ4}+ . . .

It is possible to weight each wavelength in a spectrum optically using the absorbance spectra of filter molecules. The scores are determined by reading a voltage level from a photodetector and integrating the total light through the sample and filter over a broad wavelength band. Although the scores may not be perfectly orthogonal, they are often sufficiently close to permit chemical analysis.

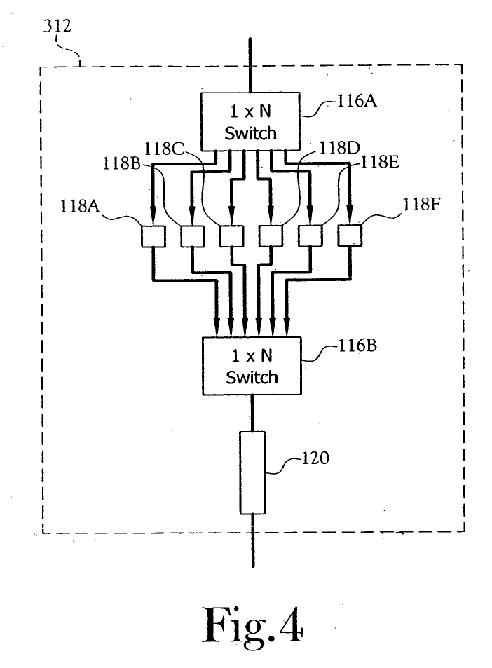

The specific use of MFCS in combination with one or more optical switches 116 uses optical fields for the interrogation and identification of preselected chemical and biological agents and constituents. In one embodiment, such as illustrated in FIG. 1, MFCS provides a very quick identification of one element or constituent of the specimen 104. In another embodiment, such as illustrated in FIGS. 2 and 3, the use of a switch 116 increases the reliability of each identification, as well as the very quick identification of many preselected constituents of the specimen 104.

FIG. 3 is a diagram of one embodiment of the present invention. The light source 102 directs a light beam 304 through a circulator 302 and into a catheter 306. In one embodiment, the catheter 306 is adapted to be inserted into a blood vessel. The catheter has a distal end for insertion into the blood vessel and a proximal end. The light beam 304 travels from the circulator 302 into the catheter 306 and out of the catheter 306 at or near its distal end. The light beam 304 is projected into a lumen and reflected back into the catheter 306, where the light beam 304 travels to the circulator 302 and the light beam 308 is directed to a switching assembly 312.

When the catheter 306 is in a blood vessel, the light beam 304 travels through the fluid in the lumen and is reflected either by the blood vessel inner wall or by other material lining the vessel such as calcified atherosclerotic plaque material or vulnerable plaque material. The returning light beam 308 directed to the switching assembly 312 includes the absorption spectrum of the fluid in the lumen and material lining the lumen wall. In this embodiment, the specimen 104 includes the fluid in the lumen and material lining the lumen wall. In the embodiment illustrated, the switching assembly 312 includes a 1×N switch 116 that has a single input of the optic signal 308 and multiple outputs, each one to a filter 118A to F. The light passes through each filter 118A to F into a corresponding photodetector 120A to F. The outputs of the photodetectors 120 are monitored by a power sensor 314 that determines the intensity of the light beam 304 after it passes through the filters 118. In one embodiment, the photodetectors 120 are responsive to the intensity of the light over the wavelength band of interest.

In one embodiment, the power sensor 314 is implemented by software running on the processor 122. In one embodiment, the analog signals from the detectors 120 are converted by an analog-to-digital converter (ADC). The software reads the input ports and stores the values corresponding to the outputs of the detectors 120. In another embodiment, the detectors 120 include ADCs that output digital signals that are input to the processor 122.

In the embodiment illustrated in FIG. 3, a light beam 304 is launched upon and reflected off the wall of the surface being examined, for instance the wall of an artery, and subsequently collected by the same optical element that launched it, and then passed back along the examination fiber through the circulator 302 to an alternate path and into an optical switch 116 where it is rapidly cycled through a number of molecular filters 118, where the spectra of that light passing through the molecular filters 118 is examined by a photodiode 120. Such an embodiment is suitable for examining, for example, the walls of the coronary arteries, where preselected molecular filters 118 examine for the white blood cell lid of an encapsulation of lipid cholesterol, lipid cholesterol, calcified cholesterol, collagen, elastin, fibrous material, necrotic tissue, or other tissue.

Any suitable type of optical fiber may be used as an examination fiber. The invention also provides embodiments including more than one optical fiber in which a separate optical fiber is used to illuminate the specimen and to collect the absorption spectrum from the specimen that results from the illumination. Thus, one system embodiment according to the invention includes an intraluminal catheter having at least one illumination fiber (e.g., one) operably linked to a light source and at least one separate spectrum-collection fiber (e.g., one). The one or more spectrum-collection fibers may be operably linked to a filter-switching and detection apparatus according to the invention. Such a system may be configured with or without a circulator.

The systems and methods of the invention are well suited for the in vivo examination of biological material. Advantageously, the methods and systems of the invention allow the detection, location, and/or diagnosis of abnormal tissue conditions of interest within a subject, such as a human patient. As indicated above, catheter-based embodiments of the invention are particularly well suited for examining material present in or associated with the lumen and walls of blood vessels. One embodiment of the invention provides a method of detecting, locating, and/or diagnosing an abnormal tissue condition in a subject by using a system and/or method of the invention to detect the presence of and/or determine the amount or concentration of at least one analyte that is characteristically associated with the abnormal tissue condition and/or is enriched in tissue having the abnormal condition. For example, an abnormal tissue condition may be associated with the presence or absence of one or more particular analytes and/or particular analyte levels, alone or in combination, versus a corresponding normal tissue condition, so that there is a characteristic profile for normal tissue and for a particular abnormal tissue condition, with respect to the particular analyte(s). A system according to the invention that includes a set of one or more molecular absorption filters having absorption spectra at least substantially specific for analyte(s) whose presence or absence or level is associated with an abnormal tissue condition can be used to examine tissue for an abnormal condition. The system may also include a computer apparatus that is programmed to determine whether a profile of constituents (analytes) that is indicative of an abnormal and/or normal tissue condition of interest is present for a particular tissue or examined section thereof. Such a system may, for example, store reference profiles for normal tissue and/or for abnormal tissue and perform comparisons between the reference profile data and diagnostic data from a subject, such as a human patient, to determine whether a sample, such as a sample region of a blood vessel, is normal or affected by the abnormal tissue condition. A single system may also be provided that permits a tissue to be examined for more than one type of abnormal tissue condition.

One embodiment of the invention provides a method for optically evaluating whether an in vivo biological sample has an abnormal condition, that includes the steps of: illuminating a biological sample in vivo with light from a light source to produce a resulting sample spectrum of light; directing at least part of the sample spectrum through at least one molecular absorption filter having an absorption spectrum at least substantially specific for a preselected atom or preselected compound, the presence, absence, or extent of the atom or compound in the sample being associated, alone or in combination with the presence, absence, or extent of one or more other analytes, with a preselected abnormal condition of the biological sample; measuring the intensity of light transmitted through the at least one molecular absorption filter; and determining, based on the amount that the molecular absorption filter reduces the intensity of light in the sample spectrum, whether the sample has the preselected abnormal condition. In one variation, the step of determining includes: determining, based on the amount that the molecular absorption filter reduces the intensity of light in the sample spectrum, whether the atom or compound is present in the sample and/or an extent to which the atom or compound is present in the sample; and determining, based at least in part on whether the atom or compound is present in the sample and/or on the extent to which the atom or compound is present in the sample, whether the sample has the preselected abnormal condition.

In another variation, the biological sample includes a blood vessel or material associated therewith and the preselected abnormal condition is an atherosclerotic condition, such as an atherosclerotic plaque condition, for example, calcified atherosclerotic plaque, and/or vulnerable plaque. In the case that the abnormal condition is an atherosclerotic condition, the or each preselected atom or preselected compound associated with the abnormal condition may, for example, be a lipid, oxidized lipid, calcium, a calcium salt, collagen, elastin, a fibrous cap material, a white blood cell constituent, or a marker compound that selectively localizes to an atherosclerotic lesion. As described earlier above, an intraluminal catheter may be used to illuminate a sample that is within or part of a blood vessel and collect the sample spectrum that results from the illumination.

A related embodiment of the invention provides a system for diagnosing an abnormal tissue condition by the optical evaluation of an in vivo sample that includes: at least one molecular absorption filter having an absorption spectrum at least substantially specific for a preselected atom or preselected compound, the presence, absence, or extent of the atom or compound in the sample being associated, alone or in combination with the presence, absence, or extent of one or more other analytes, with a preselected abnormal tissue condition; a detector configured to measure the intensity of light transmitted through the at least one molecular absorption filter; and a processor operably linked to the detector and configured to determine (by way of computer instructions), based on the amount that the molecular absorption filter reduces the intensity of light in the sample spectrum, whether the sample has the preselected abnormal tissue condition. In one variation, the processor is configured (by way of computer instructions) to determine: based on the amount that the molecular absorption filter reduces the intensity of light in the sample spectrum whether the atom or compound is present in the sample and/or an extent to which the atom or compound is present in the sample; and based at least in part on whether the atom or compound is present in the sample and/or on the extent to which the atom or compound is present in the sample, whether the sample has the preselected abnormal tissue condition.

In another variation of the system, the biological sample includes a blood vessel or material associated therewith, and the preselected abnormal tissue condition is an atherosclerotic condition, such as an atherosclerotic plaque condition, for example, calcified atherosclerotic plaque, and/or vulnerable plaque. In the case that the abnormal condition is an atherosclerotic condition, the or each preselected atom or preselected compound associated with the abnormal condition may, for example, be a lipid, oxidized lipid, calcium, a calcium salt, a fibrous cap material, a white blood cell constituent, or a marker compound that selectively localizes to an atherosclerotic lesion. In one variation, the system further includes a light-directing element configured to direct at least part of a sample spectrum of light produced by illuminating a sample with a light source through the at least one molecular absorption filter. The system may include a light source for illuminating the sample. In one variation, the system further includes an intraluminal catheter configured to deliver light from a light source to an intraluminal sample and to collect the sample spectrum obtained from illuminating the sample. Where the analysis of more than one analyte is required or desirable to determine whether the sample has the abnormal tissue condition of interest, the system may be provided with the necessary number of separate molecular absorption filters for measuring the presence of and/or the amount or concentration of each analyte.

FIG. 4 is a diagram of another embodiment of the switching assembly 312. In this embodiment, the first 1×N switch 116A directs the light beam 304 from the specimen 104 to a selected one of the filters 118A to F, and the second 1×N switch 116B receives the light beam 304 after it passes through a selected one of the filters 118A to F and passes it to a single photodetector 120. In the illustrated embodiment of the switching assembly 312, a single detector 120 is used and the two switches 116A, 116B are synchronized to select the same single filter 118A to F.

In another embodiment, the switching assembly 312 includes a rotary switch that directs the optical signal 304 through one of several filters 118 and into a photodetector 120. In one such embodiment, the optical signal 304 is directed through a rotating prism, through stationary filters 118 that are located radially around the prism, and into the photodetectors 120 on the opposite side of the filters 118. In another such embodiment, the optical signal 304 is directed through each of several filters 118 mounted on a rotating disk. The filters rotate around a stationary prism and intercept the optical signal 304 as it travels from the prism to a photodetector 120.

In another embodiment of the optical monitoring system 100, the switching assembly 312 includes an optical splitter instead of the switch 116. The optical splitter directs the optical signal 304 to several filters 118. In this embodiment, the light source 102 intensity and the transmission of the specimen 104 must be great enough to overcome the light losses of the optical splitter.

The switching speed of the switching assembly 312 may be dependent upon several factors, such as the rate of movement of the catheter 306 as it moves the light beam 302 through the lumen, the rate at which the switch 116 can select each of the filters 118, the number of filters 118, and the response time of the photodetector 120. Advantageously, one or more high-speed switching assemblies may be used according to the invention. In one embodiment, the switching assembly 312 has a cycle time of 50 milliseconds; that is, the switch 116 changes state in 50 ms. High-speed photodetectors are readily obtainable with response times on the order of 5 nanoseconds or less to accommodate the high-speed switching. In one embodiment, the switching assembly 312 includes a rotary switch that operates at 120,000 RPM. Such a rotary switch with one revolution not having any filters 118 duplicated has a sampling rate of 2000 samples per second. If the rotary switch includes duplicate filters 118, the sampling rate increases based on the number of duplicate filters 118. For example, a rotary switch with 21 filters 118 arranged as three sets of seven filters 118 has a sampling rate of 6000 samples per second with the switch rotating at 120,000 RPM.

In alternate embodiments, the switching rates between channels may, for example, range anywhere between 2 milliseconds (500 per second), maximum (operating at 2400 RPM with only 8 channels), to a minimum of 2.5 microseconds (400,000 per second), operating at 240,000 RPM, with 100 channels. The switching rate may be adjusted continuously, for example, where the motor speed is regulated by analogue electronics.

In one embodiment, the medical interventional optical monitoring system 100 operates without changing the nature, use, or habits of the interventional physician while providing the information regarding the lumen through which the catheter 306 is traveling. The system 100 also operates fast enough to completely characterize the artery for geometry and composition in real time, such that any delays in time to diagnose and time to therapy are reduced. For example, where a stent or shield is to be used to cover a vulnerable plaque can be determined as the catheter 306 travels through the lumen.

From the foregoing description, it will be recognized by those skilled in the art that a medical interventional optical monitoring system 100 has been provided. The system 100, in one embodiment, passes a light beam from a light source 102 through a catheter 306, where it is reflected back into the catheter 306 and into a switching assembly 312. The switching assembly 312, in one embodiment, includes means for directing a light beam through multiple filters 118 and into one or more photodetectors 120. When the filters 118 are molecular filters having an absorption spectrum corresponding to a compound or material of interest, the presence of that compound or material can be determined.

The invention also provides correspondingly related embodiments in which one or more molecular absorption filters are placed between outgoing light from the light source and the sample rather than between the incoming sample spectrum and the detector(s). A switching assembly, such as those described earlier, may be provided to switch between one or more molecular absorption filters and no filter (or a neutral density filter, to normalize intensity). In these embodiments, (i) light from a light source, filtered by a molecular absorption filter, is directed upon a specimen to obtain a sample absorption spectrum, for example, by reflection, and the intensity of at least part of the returned sample spectrum is measured by at least one detector; and (ii) light from the light source without such filtering is directed upon the specimen to obtain a sample absorption spectrum, for example, by reflection, and the intensity of at least part of this returned sample spectrum is measured by at least one detector. The intensity of the sample absorption spectra obtained with and without the molecular absorption filter is measured using one or more detectors. Here again, the magnitude of the difference between the measured intensity of the sample absorption spectrum with the molecular absorption filter and without the molecular absorption filter is inversely proportional to the extent (absolute or relative amount or concentration) of an analyte in the sample for which the molecular absorption filter in question is specific. These embodiments may, for example, also be provided with or performed using an intraluminal catheter that includes one or more optical fibers for illuminating a sample within a lumen, such as a blood vessel, and collecting a sample absorption spectrum so obtained.

One embodiment of the invention provides a system for optically evaluating the composition of a sample, such as a biological specimen, that includes: at least one molecular absorption filter having an absorption spectrum at least substantially specific for a preselected atom or preselected compound and configured to filter light from a light source before it illuminates a sample; a detector configured to measure the intensity of at least part of a sample absorption spectrum resulting from illuminating the sample with light from a light source that has been filtered by the molecular absorption filter and light from the light source that has not been filtered by the molecular absorption filter; and a processor operably linked to the detector and configured to determine, based on the magnitude of the difference between the measured intensity of the sample absorption spectrum with the molecular absorption filter and without the molecular absorption filter, whether the atom or compound is present in the sample and/or an extent to which the atom or compound is present in the sample. The system may further include a switching assembly, for example, as described herein, configured to switch light from the light source between the molecular absorption filter and no molecular absorption filter.

A related embodiment of the invention provides a system for optically evaluating the composition of a sample, such as a biological specimen, that includes: at least two molecular absorption filters configured to filter light from a light source before the light illuminates a sample; each molecular absorption filter having an absorption spectrum at least substantially specific for a preselected atom or preselected compound; and the absorption spectrums of at least two of the molecular absorption filters being at least substantially specific for different atoms or compounds; a light distributing element configured to distribute light from a light source through each of the at least two molecular absorption filters and through a channel without one of the molecular absorption filters (for example, without any molecular absorption filter); at least one detector, alone or together, configured to measure the intensity of at least part of a sample spectrum, for example, a reflected sample spectrum, that results from illuminating the sample with light from a light source that has been filtered (independently) by each of the molecular absorption filters and light from the light source that has not been filtered by the molecular absorption filter; and a processor operably linked to the detector and configured to determine for each of the molecular absorption filters, based on the magnitude of the difference between the measured intensity of the sample absorption spectrum with the molecular absorption filter and without the molecular absorption filter, whether the atom or compound for that filter is present in the sample and/or an extent to which the atom or compound for that filter is present in the sample.

A further embodiment of the invention provides a system for optically evaluating whether an in vivo biological sample has a preselected abnormal condition, that includes: at least one molecular absorption filter having an absorption spectrum at least substantially specific for a preselected atom or preselected compound, the presence, absence, or extent of the atom or compound in the sample being associated, alone or in combination with the presence, absence, or extent of one or more other analytes (for example, for which other molecular absorption filters are provided), with a preselected abnormal tissue condition, such as an atherosclerotic condition of a blood vessel, the molecular absorption filter being configured to filter light from a light source before it illuminates a sample; at least one detector, alone or together, configured to measure the intensity of at least part of a sample spectrum, for example, reflected sample spectrum, that results from illuminating the sample with light from a light source that has been filtered (independently) by each of the molecular absorption filters and light from the light source that has not been filtered by the molecular absorption filter(s); and a processor operably linked to the detector and configured to determine, for each of the molecular absorption filters, based on the magnitude of the difference between the measured intensity of the sample absorption spectrums obtained with light filtered by the molecular absorption filter and without filtering by the molecular absorption filter, whether the atom or compound for that filter is present in the sample and/or an extent to which the atom or compound for that filter is present in the sample; and based at least in part on whether the atom or compound is present in the sample and/or on the extent to which the atom or compound is present in the sample, whether the sample has the preselected abnormal tissue condition.

One embodiment of the invention provides a method for optically evaluating the composition of a sample, such as a biological specimen, that includes the steps of: illuminating a sample with light from a light source that is filtered by a molecular absorption filter having an absorption spectrum at least substantially specific for a preselected atom or preselected compound to produce a resulting sample absorption spectrum of light; measuring the intensity of light of the sample spectrum obtained by illuminating the sample with the light filtered by the molecular absorption filter; illuminating the sample with light from the light source that is not filtered by the molecular absorption filter (for example, without any molecular absorption filter) to produce a sample absorption spectrum; measuring the intensity of light of the sample absorption spectrum obtained by illumination without the molecular absorption filter; and determining, based on the magnitude of the difference between the measured intensity of the sample absorption spectrum with the molecular absorption filter and without the molecular absorption filter, whether the atom or compound is present in the sample and/or an extent to which the atom or compound is present in the sample.

A related embodiment of the invention provides a method for optically evaluating the composition of a sample, such as a biological specimen, that includes the steps of: illuminating a sample with light from a light source that is filtered by a first molecular absorption filter having an absorption spectrum at least substantially specific for a preselected atom or preselected compound to produce a resulting sample absorption spectrum of light; measuring the intensity of light of the sample spectrum obtained by illumination with the light filtered by the first molecular absorption filter; illuminating the sample with light from the light source that is filtered by a second molecular absorption filter having an absorption spectrum at least substantially specific for a preselected atom or preselected compound (for example, one that is different than that for the first filter) to produce a resulting sample absorption spectrum of light; measuring the intensity of light of the sample spectrum obtained by illumination with the light filtered by the second molecular absorption filter; illuminating the sample with light from the light source that is not filtered by the first and second molecular absorption filters (for example, without any molecular absorption filter) to produce a sample absorption spectrum; measuring the intensity of light of the sample absorption spectrum obtained by illumination without the molecular absorption filters; and for each of the first and second molecular absorption filters, determining, based on the magnitude of the difference between the measured intensity of the sample absorption spectrum with the molecular absorption filter and without the molecular absorption filter, whether the atom or compound for the filter is present in the sample and/or an extent to which the atom or compound for the filter is present in the sample. Related variations including still further molecular absorption filters specific for still further analytes are also provided within the scope of the embodiment.

A further embodiment of the invention provides a method for optically evaluating whether an in vivo biological sample has a preselected abnormal condition that comprises the steps of: illuminating a biological sample in vivo with light from a light source that is filtered by a molecular absorption filter to produce a sample absorption spectrum of light, the molecular absorption filter having an absorption spectrum at least substantially specific for a preselected atom or preselected compound, the presence, absence, or extent of the atom or compound in the sample being associated, alone or in combination with the presence, absence, or extent of one or more other analytes, with a preselected abnormal condition, such as an atherosclerotic condition of a blood vessel; measuring the intensity of light of the sample absorption spectrum obtained using the light filtered by the molecular absorption filter; illuminating the sample with light from the light source that is not filtered by the molecular absorption filter (for example, without any molecular absorption filter) to produce a sample absorption spectrum; measuring the intensity of light of the sample absorption spectrum obtained by illumination without the molecular absorption filter; determining, based on the magnitude of the difference between the measured intensity of the sample absorption spectrum with the molecular absorption filter and without the molecular absorption filter, whether the atom or compound is present in the sample and/or an extent to which the atom or compound is present in the sample; and determining, based at least in part on whether the atom or compound is present in the sample and/or on the extent to which the atom or compound is present in the sample, whether the sample has the preselected abnormal condition.

A further embodiment of the invention provides a method for optically evaluating whether an in vivo biological sample has a preselected abnormal condition that comprises the steps of: illuminating a biological sample in vivo with light from a light source that is filtered by a first molecular absorption filter to produce a sample spectrum of light, the first molecular absorption filter having an absorption spectrum at least substantially specific for a preselected atom or preselected compound, the presence, absence, or extent of the atom or compound in the sample being associated, alone or in combination with the presence, absence, or extent of one or more other analytes, with a preselected abnormal condition, such as an atherosclerotic condition of a blood vessel; measuring the intensity of light of the sample absorption spectrum obtained using the light filtered by the first molecular absorption filter; illuminating the biological sample in vivo with light from a light source that is filtered by a second molecular absorption filter to produce a sample spectrum of light, the second molecular absorption filter having an absorption spectrum at least substantially specific for a preselected atom or preselected compound, the presence, absence, or extent of the atom or compound in the sample being associated, alone or in combination with the presence, absence, or extent of one or more other analytes (for example, the analyte for which the first molecular absorption filter is specific), with the preselected abnormal condition; measuring the intensity of light of the sample absorption spectrum obtained using the light filtered by the second molecular absorption filter; illuminating the sample with light from a light source that is not filtered by the molecular absorption filters (for example, without any molecular absorption filter) to produce a sample absorption spectrum; measuring the intensity of light of the sample absorption spectrum obtained by illumination without the molecular absorption filter; for each of the first and second molecular absorption filters, determining, based on the magnitude of the difference between the measured intensity of the sample absorption spectrum with the molecular absorption filter and without the molecular absorption filter, whether the atom or compound for that filter is present in the sample and/or an extent to which the atom or compound for that filter is present in the sample; and determining, based at least in part on whether the atom or compound for each of the filters is present in the sample and/or on the extent to which the atom or compound for each of the filters is present in the sample, whether the sample has the preselected abnormal condition. Related variations comprising still further molecular absorption filters specific for still further analytes are also provided within the scope of the embodiment.

The invention also provides embodiments corresponding to any of the embodiments described herein above in which at least one of the molecular absorption filters is at least substantially specific for more than one preselected substance (atom(s) and/or compound(s)) of interest.

While the present invention has been illustrated by description of several embodiments, and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects, therefore, is not limited to the specific details, representative apparatus and methods, and illustrative examples shown and described. Accordingly, departures may be made from such details without departing from the spirit or scope of applicant's general inventive concept.

What is claimed is:

1. A system for optically evaluating the composition of a sample, comprising:
at least one molecular absorption filter having an absorption spectrum at least substantially specific for a preselected atom or preselected compound;
a detector configured to measure the intensity of light transmitted through the at least one molecular absorption filter; and
a processor operably linked to the detector and configured to determine, based on the amount that the molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

2. The system of claim 1 wherein the processor is further configured such that:
no reduction in said intensity indicates that the atom or compound for which the filter is specific is present in the sample.

3. The system of claim 2 wherein the processor is further configured such that:
a reduction in said intensity indicates the absence in the sample of the atom or compound for which the filter is specific.

4. The system of claim 1 wherein the processor is further configured such that:
a concentration in the sample of the atom or compound for which the molecular absorption filter is specific is determined in inverse proportion to the size of the amount of a non-zero reduction in said intensity.

5. The system of claim 1, further comprising the light source.

6. The system of claim 5 wherein the light source comprises a multi-wavelength light source.

7. The system of claim 5 wherein the light source comprises an at least substantially continuous spectrum light source.

8. The system of claim 1, further comprising means for examining a sample within or part of a blood vessel using the system.

9. The system of claim 1, further comprising an intraluminal catheter adapted for insertion into a blood vessel, the catheter comprising at least one optical fiber, wherein an optical fiber is operably linkable to a light source to illuminate the sample and an optical fiber is configured to collect an absorption spectrum resulting from illumination of the sample.

10. The system of claim 1 wherein the preselected atom or preselected compound for at least one of the molecular absorption filters is associated with a preselected abnormal condition of blood vessels.

11. The system of claim 1, further comprising:
a light distributing element configured to direct at least part of a sample spectrum of light produced by illuminating a sample with a light source through the at least one molecular absorption filter.

12. A system for optically evaluating the composition of a sample, comprising:
at least two molecular absorption filters;
each molecular absorption filter having an absorption spectrum at least substantially specific for a preselected atom or preselected compound; and
the absorption spectrums of at least two of the molecular absorption filters being at least substantially specific for different atoms or compounds;
a light distributing element configured to distribute at least part of a sample spectrum of light produced by illuminating a sample with a light source through each of the at least two molecular absorption filters;
at least one detector configured to measure the intensity of light transmitted through each of the molecular absorption filters; and
a processor operably linked to the detector and configured to determine, based on the amount that a molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

13. The system of claim 12 wherein the processor is further configured such that:
no reduction in said intensity indicates that the atom or compound for which the filter is specific is present in the sample.

14. The system of claim 13 wherein the processor is further configured such that:
a reduction in said intensity indicates the absence in the sample of the atom or compound for which the filter is specific.

15. The system of claim 12 wherein the processor is further configured such that:
the concentration in the sample of the atom or compound for which the molecular absorption filter is specific is determined in inverse proportion to the size of the amount of a non-zero reduction in said intensity.

16. The system of claim 12, further comprising the light source.

17. The system of claim 16 wherein the light source comprises a multi-wavelength light source.

18. The system of claim 16 wherein the light source comprises an at least substantially continuous spectrum light source.

19. The system of claim 12 wherein the distributing element comprises a switching assembly.

20. The system of claim 12 wherein the distributing element comprises a rotary switch.

21. The system of claim 12 wherein the distributing element comprises an optical splitter.

22. The system of claim 12, further comprising means for examining a sample within or part of a blood vessel using the system.

23. The system of claim 12, further comprising an intraluminal catheter adapted for insertion into a blood vessel, the catheter comprising at least one optical fiber, wherein an optical fiber is operably linkable to a light source to illuminate the sample and an optical fiber is configured to collect an absorption spectrum resulting from illumination of the sample.

24. The system of claim 12 wherein the preselected atom or preselected compound for at least one of the molecular absorption filters is associated with a preselected abnormal condition of blood vessels.

25. A system for optically evaluating the composition of a sample, comprising:
at least one molecular absorption filter having an absorption spectrum at least substantially specific for a preselected atom or preselected compound;
at least one light intensity detector configured to measure the intensity transmitted through the at least one filter; and
determining means for determining, based on the amount that a molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

26. The system of claim 25 wherein the determining means determine that the atom or compound for which the filter is specific is present in the sample if there is at least no substantial reduction in said intensity.

27. The system of claim 25 wherein the determining means determine that the atom or compound for which the filter is specific is absent in the sample if there is a reduction in said intensity.

28. The system of claim 25 wherein the determining means determine a concentration in the sample of the atom or compound for which the molecular absorption filter is specific in inverse proportion to the size of the amount of a non-zero reduction in said intensity.

29. The system of claim 25, further comprising the light source.

30. The system of claim 29 wherein the light source comprises means for illuminating the sample with light of greater than one wavelength.

31. The system of claim 25 wherein the sample is within or part of a blood vessel.

32. The system of claim 25, further comprising means for examining a sample within or part of a blood vessel.

33. The system of claim 25, further comprising:
means for directing at least part of a sample spectrum produced by illuminating a sample with a light source through the at least one molecular absorption filter.

34. A system for optically evaluating the composition of a sample, comprising:
at least two molecular absorption filters;
each filter having an absorption spectrum at least substantially specific for a preselected atom or preselected compound; and
the absorption spectrums of at least two of the filters being at least substantially specific for different atoms or compounds;
means for distributing at least part of a sample spectrum produced by illuminating a sample with a light source through each of the at least two molecular absorption filters;
at least one light intensity detector configured to measure the intensity of light transmitted through each of the filters; and
determining means for determining, based on the amount that a molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

35. The system of claim 34 wherein the determining means determine that the atom or compound for which the filter is specific is present in the sample if there is at least no substantial reduction in said intensity.

36. The system of claim 35 wherein the determining means further determine that the atom or compound for which the filter is specific is absent in the sample if there is a reduction in said intensity.

37. The system of claim 34 wherein the determining means determine a concentration in the sample of the atom or compound for which the molecular absorption filter is specific in inverse proportion to the size of the amount of a non-zero reduction in said intensity.

38. The system of claim 34, further comprising the light source.

39. The system of claim 38 wherein the light source comprises means for illuminating the sample with light of greater than one wavelength.

40. The system of claim 34, further comprising means for examining a sample within or part of a blood vessel.

41. A method for optically evaluating the composition of a sample, comprising the steps of:
illuminating a sample with light from a light source to produce a resulting sample spectrum of light;
directing at least part of the sample spectrum through at least one molecular absorption filter having an absorption spectrum at least substantially specific for a preselected atom or preselected compound;
measuring the intensity of light transmitted through the at least one molecular absorption filter; and
determining, based on the amount that a molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

42. The method of claim 41 wherein the step of determining further comprises:
determining that the atom or compound for which the filter is specific is present in the sample if there is at least no substantial reduction in said intensity.

43. The method of claim 42 wherein the step of determining further comprises:
determining that the atom or compound for which the filter is specific is absent in the sample if there is a reduction in said intensity.

44. The method of claim 41 wherein the step of determining further comprises:
determining a concentration in the sample of the atom or compound for which the molecular absorption filter is specific in inverse proportion to the size of the amount of a non-zero reduction in said intensity.

45. The method of claim 41 wherein the step of illuminating the sample comprises illuminating the sample with greater than one wavelength of light.

46. The method of claim 41 wherein the light source comprises an at least substantially continuous spectrum light source.

47. The method of claim 41 wherein the sample is within or part of a blood vessel.

48. The method of claim 47 wherein the preselected atom or preselected compound for at least one of the molecular absorption filters is associated with a preselected abnormal condition of blood vessels.

49. A method for optically evaluating the composition of a sample, comprising:
illuminating a sample with light from a light source to produce a resulting sample spectrum of light;
directing the sample spectrum through each of at least two molecular absorption filters;
each molecular absorption filter having an absorption spectrum at least substantially specific for a preselected atom or preselected compound; and
the absorption spectrums of at least two of the molecular absorption filters being at least substantially specific for different atoms or compounds;
measuring the intensity of light transmitted through each of the molecular absorption filters; and
for each of the molecular absorption filters, determining, based on the amount that the molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

50. The method of claim 49 wherein the step of determining further comprises:
determining that the atom or compound for which the filter is specific is present in the sample if there is at least no substantial reduction in said intensity.

51. The method of claim 50 wherein the step of determining further comprises:
determining that the atom or compound for which the filter is specific is absent in the sample if there is a reduction in said intensity.

52. The method of claim 49 wherein the step of determining further comprises:
determining a concentration in the sample of the atom or compound for which the molecular absorption filter is specific in inverse proportion to the size of the amount of a non-zero reduction in said intensity.

53. The method of claim 49 wherein the step of illuminating the sample comprises illuminating the sample with greater than one wavelength of light.

54. The method of claim 49 wherein the light source comprises an at least substantially continuous spectrum light source.

55. The method of claim 49 wherein the sample is within or part of a blood vessel.

56. The method of claim 49 wherein the preselected atom or preselected compound is characteristic of blood vessels or material associated with blood vessels.

57. The method of claim 49 wherein the step of directing the sample spectrum through each of at least two molecular absorption filters is performed sequentially with respect to each filter.

58. The method of claim 49 wherein the step of directing the sample spectrum through each of at least two molecular absorption filters comprises sequentially switching the sample spectrum between at least two of the filters.

59. The method of claim 49 wherein the step of directing the sample spectrum through each of the at least two molecular absorption filters comprises splitting the sample spectrum between at least two of the molecular absorption filters.

60. A method for optically evaluating the composition of a sample, comprising:
illuminating a sample with light from a light source to produce a resulting sample spectrum of light;
a step for directing the sample spectrum through at least one molecular absorption filter having an absorption spectrum at least substantially specific for a preselected atom or preselected compound;
measuring the intensity of light transmitted through the at least one molecular absorption filter; and
a step for determining, based on the amount that a molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

61. The method of claim 60 wherein the light from the light source comprises greater than one wavelength of light.

62. The method of claim 60 wherein the light source comprises an at least substantially continuous spectrum light source.

63. The method of claim 60 wherein the sample is within or part of a blood vessel.

64. The method of claim 63 wherein the preselected atom or preselected compound for at least one of the molecular absorption filters is associated with a preselected abnormal condition of blood vessels.

65. A method for optically evaluating the composition of a sample, comprising:
illuminating a sample with light from a light source to produce a resulting sample spectrum of light;
a step for directing the sample spectrum through each of at least two molecular absorption filters;
each filter having an absorption spectrum at least substantially specific for a preselected atom or preselected compound; and
the absorption spectrums of at least two of the molecular absorption filters being at least substantially specific for different atoms or compounds;
for each of the molecular absorption filters, measuring the intensity of light transmitted through the molecular absorption filter; and
for each of the molecular absorption filters, a step for determining, based on the amount that the molecular absorption filter reduces the intensity of light in the sample spectrum, (i) whether the atom or compound for that filter is present in the sample; or (ii) an extent to which the atom or compound for that filter is present in the sample; or (iii) both (i) and (ii).

66. The method of claim 65 wherein the sample is within or part of a blood vessel.

67. The method of claim 66 wherein the preselected atom or preselected compound for at least one of the molecular absorption filters is associated with a preselected abnormal condition of blood vessels.

68. The method of claim 65 wherein the light from the light source comprises greater than one wavelength of light.

69. The method of claim 65 wherein the light source comprises an at least substantially continuous spectrum light source.

70. A method of for optically evaluating the composition of a sample, comprising:
illuminating a sample with light from a light source, filtered by a molecular absorption filter; and
analyzing a sample absorption spectrum returned from the sample.

71. The method according to claim 70 wherein the step of analyzing includes determining whether an atom or compound is present in the sample.
